(19) United States
(12) Patent Application Publication
(10) Pub. No.: US 2022/0185764 A1
(43) Pub. Date: Jun. 16, 2022

(54) Resveratrol derivative and anti-fibrotic method using the same

(71) Applicant: TAIZHOU HUAYUAN MEDICINAL TECH CO. LTD., Taizhou (CN)

(72) Inventors: Bohua ZHONG, Taizhou (CN); Hongwu LI, Taizhou (CN); Jiajun YANG, Taizhou (CN)

(21) Appl. No.: 17/118,318

(22) Filed: Dec. 10, 2020

(51) Int. Cl. C07C 69/708; A61P 1/16; A61P 11/00; A61K 9/00

(52) U.S. Cl. CPC C07C 69/708; A61K 9/0019; A61P 11/00; A61P 1/16

(57) ABSTRACT

A resveratrol derivative of the compound of formula I or a pharmaceutically acceptable salt thereof are effective for treating a fibrotic disease.

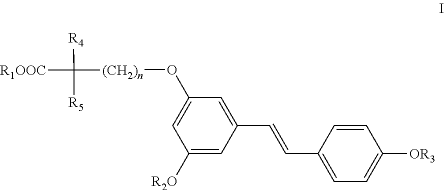

In formula I, R₁ is hydrogen or C₁-C₄ alkyl, R₂ and R₃ are each independently C₁-C₃ alkyl or C₁-C₃ deuterated alkyl, and R₂ and R₃ are the same or different; the C₁-C₄ alkyl or C₁-C₃ alkyl is optionally substituted with one or more substituents each independently selected from the group consisting of halogen (for example, fluorine, chlorine, bromine, iodine), methyl, hydroxyl, ethyl, amino, methoxy, and nitro; and R₄ and R₅ are substituted or unsubstituted C₁-C₃ alkyl. The fibrotic disease is selected from the group consisting of liver fibrosis, lung fibrosis, kidney fibrosis, and cardiac fibrosis.

### TECHNICAL FIELD OF THE INVENTION

The present invention relates to anti-fibrotic resveratrol derivatives, pharmaceutically acceptable salts thereof, pharmaceutical compositions containing these compounds as active ingredients, and a method for treating a fibrotic disease comprising the step of administering to a subject in need thereof an effective amount of the resveratrol derivative, pharmaceutically acceptable salt thereof, or the pharmaceutical composition.

### BACKGROUND OF THE INVENTION

The fibrosis of human tissues or organs such as liver fibrosis, lung fibrosis, kidney fibrosis, and cardiac fibrosis, is caused by the deposition of extracellular matrix with collagen as the main component, and is the result of continuous damage to related organs or tissues. A variety of signal factors cause continuous tissue damage, including transforming growth factor-β1 (TGF-β1), connective tissue growth factor (CTG F), platelet-derived growth factor (PDGF), vascular endothelial growth factor (VEGF), fibroblasts growth factor (FGF) and the like.

### SUMMARY OF THE INVENTION

The present invention provides a resveratrol derivative of formula I or a pharmaceutically acceptable salt thereof:

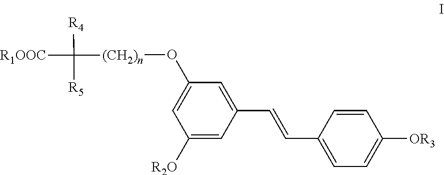

in formula I, R₁ is hydrogen or C₁-C₄ alkyl, R₂ and R₃ are each independently C₁-C₃ alkyl or C₁-C₃ deuterated alkyl, R₂ and R₃ are the same or different; the C₁-C₄ alkyl or C₁-C₃ alkyl is optionally substituted with one or more substituents each independently selected from the group consisting of halogen (for example, fluorine, chlorine, bromine, iodine), methyl, hydroxyl, ethyl, amino, methoxy, and nitro; R₄ and R₅ are each independently substituted or unsubstituted C₁-C₃ alkyl; and n is an integer of 0-5.

In an embodiment of the invention, the resveratrol derivative of formula I has the following formula I-0:

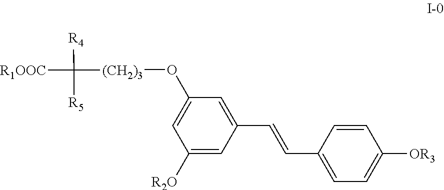

in formula I-0, R₁ is hydrogen or C₁-C₄ alkyl, R₂ and R₃ are each independently C₁-C₃ alkyl or C₁-C₃ deuterated alkyl, R₂ and R₃ are the same or different; the C₁-C₄ alkyl or C₁-C₃ alkyl is optionally substituted with one or more substituents each independently selected from the group consisting of halogen (for example, fluorine, chlorine, bromine, iodine), methyl, hydroxyl, ethyl, amino, methoxy, and nitro; and R₄ and R₅ are each independently substituted or unsubstituted C₁-C₃ alkyl.

In an embodiment of the invention, the resveratrol derivative of formula I has the following formula I-1:

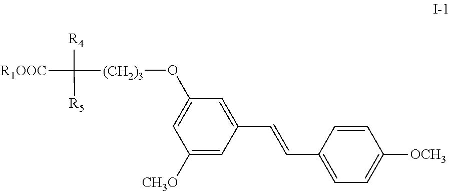

in formula I-1, R₁ is hydrogen or C₁-C₄ alkyl; wherein, the C₁-C₄ alkyl is optionally substituted with one or more substituents each independently selected from the group consisting of halogen (for example, fluorine, chlorine, bromine, iodine), methyl, hydroxyl, ethyl, amino, methoxy, and nitro; R₄ and R₅ are each independently substituted or unsubstituted C₁-C₃ alkyl.

In an embodiment of the invention, the resveratrol derivative of formula I has the following formula I-2:

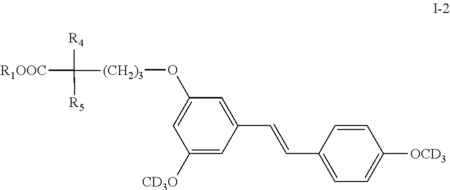

in formula I-2, R₁ is hydrogen or C₁-C₄ alkyl; wherein, the C₁-C₄ alkyl is optionally substituted with one or more substituents each independently selected from the group consisting of halogen (for example, fluorine, chlorine, bromine, iodine), methyl, hydroxyl, ethyl, amino, methoxy, and nitro; and R₄ and R₅ are each independently substituted or unsubstituted C₁-C₃ alkyl. In an embodiment of the invention, the resveratrol derivative of formula I has the following formula I-3:

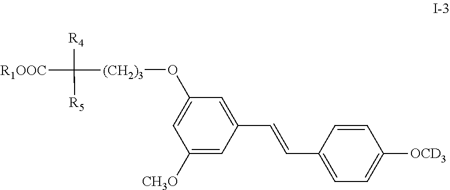

in formula I-3, R₁ is hydrogen or C₁-C₄ alkyl; wherein, the C₁-C₄ alkyl is optionally substituted with one or more substituents each independently selected from the group consisting of: halogen (for example, fluorine, chlorine, bromine, iodine), methyl, hydroxyl, ethyl, amino, methoxy, and nitro; and R₄ and R₅ are each independently substituted or unsubstituted C₁-C₃ alkyl. In an embodiment of the invention, the resveratrol derivative of formula I has the following formula I-4:

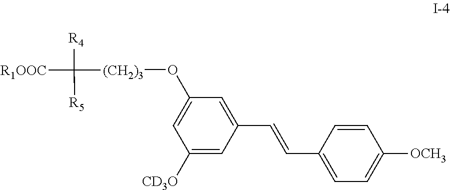

in formula I-4, R₁ is hydrogen or C₁-C₄ alkyl; wherein, the C₁-C₄ alkyl is optionally substituted with one or more substituents each independently selected from the group consisting of halogen (for example, fluorine, chlorine, bromine, iodine), methyl, hydroxyl, ethyl, amino, methoxy, and nitro; and R₄ and R₅ are each independently substituted or unsubstituted C₁-C₃ alkyl.

In an embodiment of the invention, the resveratrol derivative of formula I has the structure as shown in the following table:

| | |
|---|---|
| 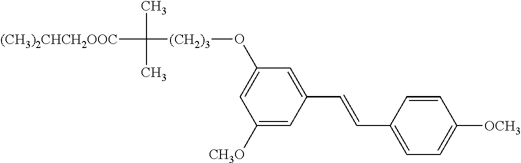 | I-1a |
| 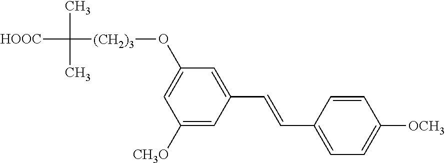 | I-1b |
| 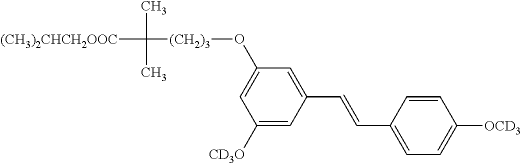 | I-2a |
| 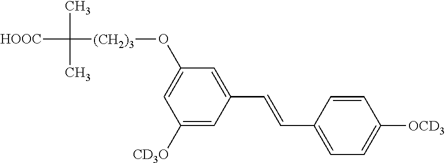 | I-2b |
| 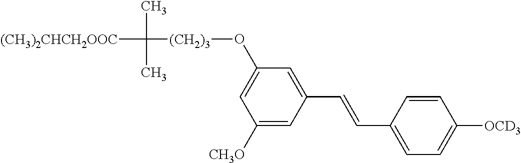 | I-3a |
| 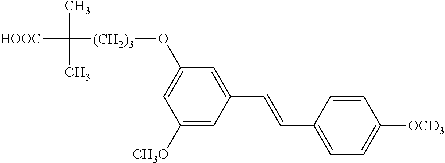 | I-3b |
| 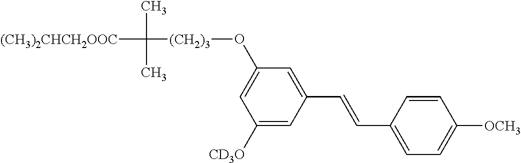 | I-4a |
| 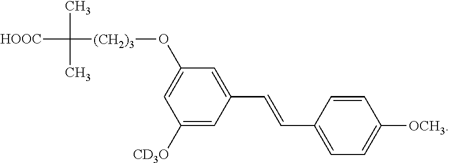 | I-4b |

Further, the present invention provides a method for treating a fibrotic disease in a subject in need thereof, comprising the step of administrating to the subject an effective amount of a resveratrol derivative of formula I or a pharmaceutically acceptable salt thereof:

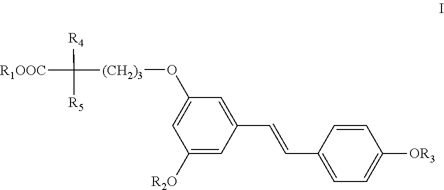

in formula I, R₁ is hydrogen or C₁-C₄ alkyl, R₂ and R₃ are each independently C₁-C₃ alkyl or C₁-C₃ deuterated alkyl, R₂ and R₃ are the same or different; wherein, the C₁-C₄ alkyl or C₁-C₃ alkyl is optionally substituted with one or more substituents each independently selected from the group consisting of halogen (for example, fluorine, chlorine, bromine, iodine), methyl, hydroxyl, ethyl, amino, methoxy, and nitro; R₄ and R₅ are each independently substituted or unsubstituted C₁-C₃ alkyl; and the fibrotic disease is selected from the group consisting of liver fibrosis, lung fibrosis, kidney fibrosis and cardiac fibrosis.

The present invention also provides a method for treating a fibrotic disease in a subject in need thereof, comprising the step of administrating to the subject an effective amount of a resveratrol derivative of formula I-0 or a pharmaceutically acceptable salt thereof:

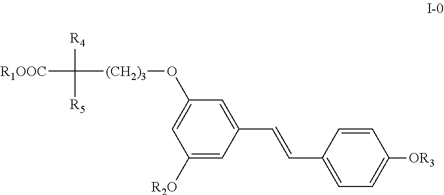

in formula I-0, R₁ is hydrogen or C₁-C₄ alkyl, R₂ and R₃ are each independently C₁-C₃ alkyl or C₁-C₃ deuterated alkyl, R₂ and R₃ are the same or different; the C₁-C₄ alkyl or C₁-C₃ alkyl is optionally substituted with one or more substituents each independently selected from the group consisting of halogen (for example, fluorine, chlorine, bromine, iodine), methyl, hydroxyl, ethyl, amino, methoxy, and nitro; and R₄ and R₅ are each independently substituted or unsubstituted C₁-C₃ alkyl; and the fibrotic disease is selected from the group consisting of liver fibrosis, lung fibrosis, kidney fibrosis and cardiac fibrosis.

More specifically, the present invention also provides a method for treating a fibrotic disease in a subject in need thereof, comprising the step of administrating to the subject an effective amount of a resveratrol derivative of formula I-1 or a pharmaceutically acceptable salt thereof:

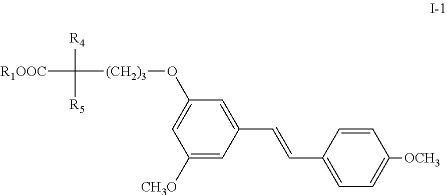

in formula I-1, R₁ is hydrogen or C₁-C₄ alkyl; wherein, the C₁-C₄ alkyl is optionally substituted with one or more substituents each independently selected from the group consisting of halogen (for example, fluorine, chlorine, bromine, iodine), methyl, hydroxyl, ethyl, amino, methoxy, and nitro; R₄ and R₅ are each independently substituted or unsubstituted C₁-C₃ alkyl; and the fibrotic disease is selected from the group consisting of liver fibrosis, lung fibrosis, kidney fibrosis and cardiac fibrosis.

The present invention also provides a method for treating a fibrotic disease in a subject in need thereof, comprising the step of administrating to the subject an effective amount of a resveratrol derivative of formula I-2 or a pharmaceutically acceptable salt thereof:

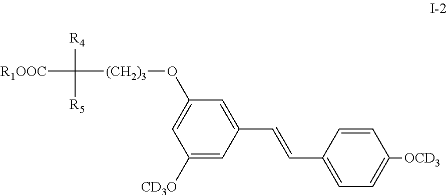

in formula I-2, R₁ is hydrogen or C₁-C₄ alkyl; wherein, the C₁-C₄ alkyl is optionally substituted with one or more substituents each independently selected from the group consisting of halogen (for example, fluorine, chlorine, bromine, iodine), methyl, hydroxyl, ethyl, amino, methoxy, and nitro; R₄ and R₅ are each independently substituted or unsubstituted C₁-C₃ alkyl; and the fibrotic disease is selected from the group consisting of liver fibrosis, lung fibrosis, kidney fibrosis and cardiac fibrosis.

The present invention also provides a method for treating a fibrotic disease in a subject in need thereof, comprising the step of administrating to the subject an effective amount of a resveratrol derivative of formula I-3 or a pharmaceutically acceptable salt thereof:

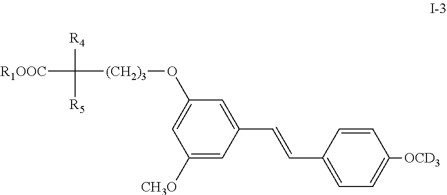

in formula I-3, R₁ is hydrogen or C₁-C₄ alkyl; wherein, the C₁-C₄ alkyl is optionally substituted with one or more substituents each independently selected from the group consisting of: halogen (for example, fluorine, chlorine, bromine, iodine), methyl, hydroxyl, ethyl, amino, methoxy, and nitro; R₄ and R₅ are each independently substituted or unsubstituted C₁-C₃ alkyl; and the fibrotic disease is selected from the group consisting of liver fibrosis, lung fibrosis, kidney fibrosis and cardiac fibrosis.

The present invention also provides a method for treating a fibrotic disease in a subject in need thereof, comprising the step of administrating to the subject an effective amount of a resveratrol derivative of formula I-4 or a pharmaceutically acceptable salt thereof:

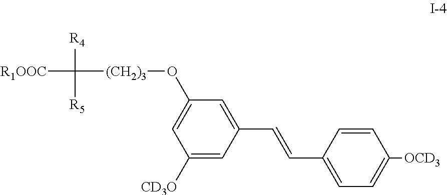

in formula I-4, R₁ is hydrogen or C₁-C₄ alkyl; wherein, the C₁-C₄ alkyl is optionally substituted with one or more substituents each independently selected from the group consisting of halogen (for example, fluorine, chlorine, bromine, iodine), methyl, hydroxyl, ethyl, amino, methoxy, and nitro; R₄ and R₅ are each independently substituted or unsubstituted C₁-C₃ alkyl; and the fibrotic disease is selected from the group consisting of liver fibrosis, lung fibrosis, kidney fibrosis and cardiac fibrosis.

The present invention also provides a method for treating a fibrotic disease in a subject in need thereof, comprising the step of administrating to the subject an effective amount of a resveratrol derivative of the following structure as shown in the above table or a pharmaceutically acceptable salt thereof, wherein, the fibrotic disease is selected from the group consisting of liver fibrosis, lung fibrosis, kidney fibrosis and cardiac fibrosis.

The present invention also provides a pharmaceutical composition comprising a compound of the above formula I, formula I-1, formula I-2, formula I-3, formula I-4, formula I-1a, formula I-1b, formula I-2a, formula I-2b, formula I-3a, formula I-3b, formula I-4a, formula I-4b, or a pharmaceutically acceptable salt thereof, and suitable pharmaceutical carriers or excipients.

The present invention also provides a method for treating a fibrotic disease in a subject in need thereof, comprising the step of administrating to the subject an effective amount of the above composition, wherein the fibrotic disease is selected from the group consisting of liver fibrosis, pulmonary fibrosis, kidney fibrosis and cardiac fibrosis.

The pharmaceutical composition of the present invention may be a solution, tablet, capsule or injection; and the pharmaceutical composition may be administered by injection or orally.

### DETAILED DESCRIPTION OF THE INVENTION

The present invention may be further described by reference to the following examples, however, the scope of the present invention is not limited to the following examples. Those skilled in the art will appreciate that various changes and modifications may be made to the present invention without departing from the spirit and scope of the present invention. The deuterium abundance of deuterated iodomethane used in all examples is greater than 99.0%, unless otherwise specified.

When R₂ and R₃ are the same, the compound of formula I in the present invention can be prepared according to the following synthetic route:

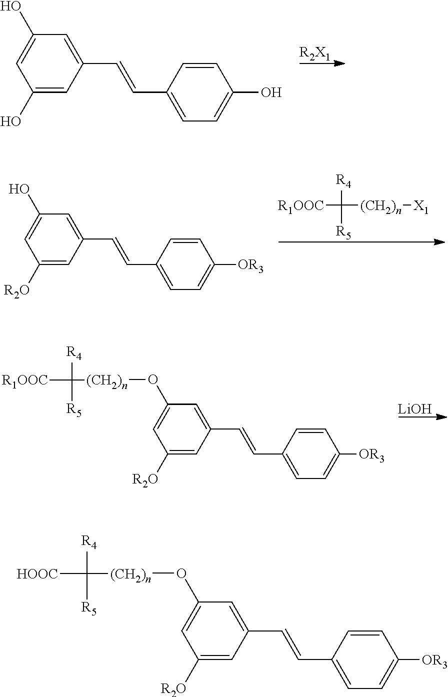

Resveratrol is used as the raw material and reacted with an alkyl halide in the presence of potassium carbonate to obtain 3,4′-dialkoxy-5-hydroxy-(E)-stilbene; 3,4′-dialkoxy-5-hydroxy-(E)-stilbene is reacted with an ester of haloalkyl acid to obtain the target compound with R₁ =alkyl, which is then hydrolyzed in the presence of lithium hydroxide to obtain the target compound with R₁=H.

In the formulas in the above synthetic route, R₁ is hydrogen or C₁-C₄ alkyl, R₂ and R₃ are the same and both are C₁-C₃ alkyl or C₁-C₃ deuterated alkyl; the C₁-C₄ alkyl or C₁-C₃ alkyl is optionally substituted with one or more substituents each independently selected from group consisting of halogen (for example, fluorine, chlorine, bromine, iodine), methyl, hydroxyl, ethyl, amino, methoxy, and nitro; R₄ and R₅ are substituted or unsubstituted C₁-C₃ alkyl; and n is an integer of 0-5.

When R₂ and R₃ are different, the compound of formula I in the present invention can be prepared according to the following synthetic route:

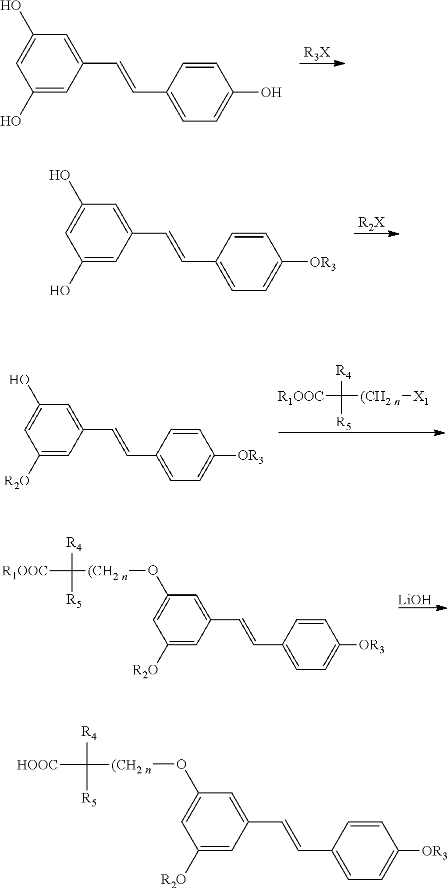

Resveratrol is used as the raw material and reacted with an alkyl halide R₃X in the presence of potassium carbonate to obtain 4′-R₃-oxy-3,5-dihydroxy-(E)-stilbene; 4′-R₃-oxy-3,5-dihydroxy-(E)-stilbene is reacted with alkyl halide R₂X in the presence of potassium carbonate to obtain 3-R₂-oxy-4′-R₃-oxy-5-hydroxy-(E)-stilbene; 3-R₂-oxy-4′-R₃-oxy-5-hydroxy-(E)-stilbene is reacted with an ester of haloalkyl acid to obtain the target compound with R₁ =alkyl, which is then hydrolyzed in the presence of lithium hydroxide to obtain the target compound with R₁=H.

In the formulas in the above synthetic route, R₁, R₂ and R₃ have the same definitions as described above.

### Reference example 1

### Preparation of Compounds I-1a and I-1b

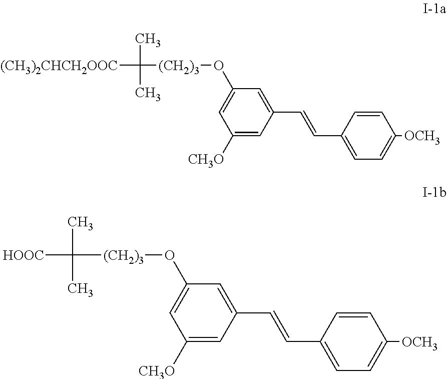

### Reference example 1.1

### 3,4′-dimethoxy-5-hydroxy-(E)-stilbene (i-1)

20 g resveratrol was dissolved in 400 mL acetone, and then 30.3 g K₂CO₃ and 6.66 mL CH₃I were successively added. The reaction mixture was heated to reflux for 10 hours under nitrogen protection. After the heating was stopped, the reaction mixture was cooled down, and filtered. The filter cake was washed with acetone, and the filtrate and the acetone washing solution were combined. The combined solution was concentrated, separated by 200-300 mesh silica gel column chromatography and eluted with a mixed solvent of petroleum ether:ethyl acetate:acetic acid (8.5:1.5:0.16) to obtain 4.9 g 3,4′-dimethoxy-5 -hydroxy-(E)-stilbene (i-1).

### Reference Example 1.2

### 3,4′-dimethoxy-5-(4-methyl-4-isobutoxycarbonylpentyloxy)-(E)-stilbene (I-1a)

2.6 g i-1 was dissolved in 40 mL dry dimethylformamide, and then 4.0 g K₂CO₃, was added. The reaction mixture was heated to 55° C. under stirring and nitrogen protection and then 3.2 g isobutyl 5-chloro-2,2-dimethylvalerate was added dropwise. After the completion of the addition, the reaction mixutre was further maitained at 55° C. for 18 h. After the heating was stopped and the reaction mixture was cooled down, 100 mL ethyl acetate was added. The mixture was washed with 1N hydrochloric acid for 3 times, 50 mL/time. The organic layer was dried over anhydrous Na₂SO₄, filtered, and concentrated. The residual was separated by 200-300 mesh silica gel column chromatography and eluted with a mixed solvent of petroleum ether:ethyl acetate:acetic acid (8:2:0.2) to obtain 1.27 g colorless oil of I-1a.

Reference Example 1.3

### 3,4′-dimethoxy-5-(4-methyl-4-carboxypentyloxy)-(E)-stilbene (I-1b)

0.91 g I-1a was dissolved in 20 mL methanol. 4 g LiOH.H₂O was prepared into 15 mL aqueous solution. The aqueous solution of LiOH.H₂O was then slowly added dropwise to the methanol solution of I-1a under stirring. The reaction mixture was stirred at room temperature for 4 days. TLC was used to monitor the reaction until the spots of the raw materials disappeared completely. The reaction solution was added with 80 mL water, adjusted to pH=3-4 with 3M hydrochloric acid, and then extracted with ethyl acetate for 3 times (20 mL/time). The extracts were combined, and washed with water to neutral. The organic layer was dried over anhydrous Na₂SO₄, filtered and evaporated to dryness under reduced pressure to obtain 0.62 g of I-1b as a pale yellow solid. ¹H-NMR (d6-DMSO,δ ppm): 1.12 (s, 6H), 1.60-1.67 (m, 4H), 3.77 (m, 6H), 3.95-3.97 (m, 2H), 6.36 (m, 1H), 6.72 (d, 2H), 6.94-7.03 (m, 3H), 7.22 (d, 1H), 7.53 (d, 2H), 12.03 (s, 1H).

### EXAMPLE 1

### Preparation of compounds I-2a and I-2b

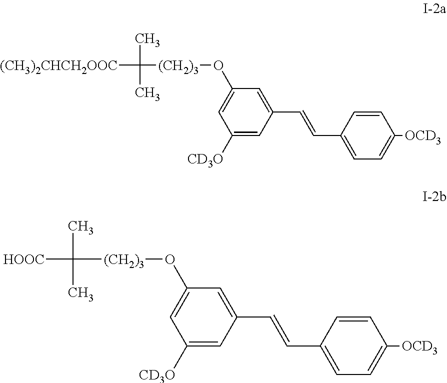

### Example 1.1

### 3,4′-bis(trideuterated methoxy)-5-hydroxy-(E)-stilbene (i-2)

10 g resveratrol was dissolved in 200 mL acetone, and then 15 g K₂CO₃ and 3.3 mL CD₃I were successively added. The reaction mixture was heated to reflux for 10 hours under nitrogen protection. After the heating was stopped, the reaction mixture was cooled down and filtered. The filter cake was washed with acetone, and the filtrate and the acetone washing solution were combined. The combined solution was concentrated, separated by 200-300 mesh silica gel column chromatography and eluted with a mixed solvent of petroleum ether:ethyl acetate:acetic acid (8.5:1.5:0.16) to obtain 4.1 g 3,4′-bis(trideuterated methoxy)-5-hydroxy-(E)-stilbene (i-2).

### Example 1.2

3,4′-bis-[(trideuterated methyl)-oxy]-5-(4-methyl-4-isobutoxycarbonylpentyloxy)-(E)-stilbene (I-2a)

Referring to the method of reference example 1.2, i-2 was reacted with isobutyl 5-chloro-2,2-dimethylvalerate, and the obtained reaction mixture was separated by silica gel column chromatography to obtain a colorless oil of I-2a with a yield of 53%.

### Example 1.3

### 3,4′-bis-[(trideuterated methyl)-oxy]-5-(4-methyl-4-carboxypentyloxy)-(E)-stilbene (I-2b)

Referring to the method of reference example 1.3, I-2a was reacted with LiOH to obtain I-2b with a yield of 78%. 1H-NMR (d₆-DMSO, δ ppm): 1.14 (s, 6H), 1.61-1.68 (m, 4H), 3.95-3.98 (m, 2H), 6.38 (m, 1H), 6.95-7.05 (m, 3H)), 7.24 (d, 1H), 7.55 (d, 2H), 12.10 (s, 1H).

### EXAMPLE 2

### Preparation of Compound I-3a and I-3b

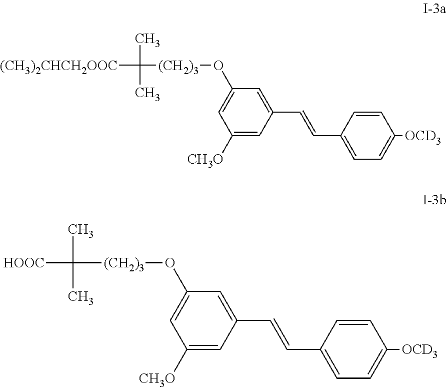

### Example 2.1

### 4′-trideuterated methoxy-3,5-dihydroxy-(E)-stilbene (i-3)

2.0 g resveratrol was dissolved in 40 mL acetone, and 3 g K₂CO₃ and 0.3 mL CD₃I were added successively. The reaction mixture was heated to reflux for 10 hours under nitrogen protection. After the heating was stopped, the reaction mixture was cooled down and filtered. The filter cake was washed with acetone and the filtrate and the acetone washing solution were combined. The combined solution was concentrated, separated by 200-300 mesh silica gel column chromatography and eluted with a mixed solvent of petroleum ether:ethyl acetate:acetic acid (8.5:1.5:0.16) to obtain 1.2 g 4′-trideuterated methoxy-3,5-dihydroxy-(E)-stilbene (i-3).

### Example 2.2

### 3-methoxy-4′-trideuterated methoxy-5-hydroxy-(E)-stilbene (i-4)

1.0 g i-3 was dissolved in 20 mL acetone, and 1 g K₂CO₃ and 0.15 mL CD₃I were added successively. The reaction mixture was heated to reflux for 10 hours under nitrogen protection. After the heating was stopped, the reaction mixture was cooled down and filtered. The filter cake was washed with acetone, and the filtrate and the acetone washing solution were combined. The combined solution was concentrated, separated by 200-300 mesh silica gel column chromatography and eluted with a mixed solvent of petroleum ether:ethyl acetate:acetic acid (8.5:1.5:0.16) to obtain 0.45 g 3-methoxy-4′-trideuterated methoxy-5-hydroxy-(E)-stilbene (i-4).

### Example 2.3

### 3-methoxy-4′-trideuterated methoxy-5-(4-methyl-4-isobutoxycarbonylpentyloxy)-(E)-stilbene (I-3a)

Referring to the method of reference example 1.2, i-4 was reacted with isobutyl 5-chloro-2,2-dimethylvalerate, and the obtained reaction mixture was separated by silica gel column chromatography to obtain a colorless oil of I-3a with a yield of 47%.

### Example 2.4

### 3-methoxy-4′-trideuterated methoxy-5-(4-methyl-4-carboxypentyloxy)-(E)-stilbene (I-3b)

Referring to the method of reference example 1.3, I-3a was reacted with LiOH to prepare I-3b with a yield of 80%. ¹H-NMR (d₆-DMSO, δ ppm): 1.13 (s, 6H), 1.61-1.69 (m, 4H), 3.78 (s, 3H), 3.96-3.98 (m, 2H), 6.37 (m, 1H), 6.95-7.04 (m, 3H), 7.23 (d, 1H), 7.54 (d, 2H), 12.13 (s, 1H).

### EXAMPLE 3

### Preparation of Compounds I-4a and I-4b

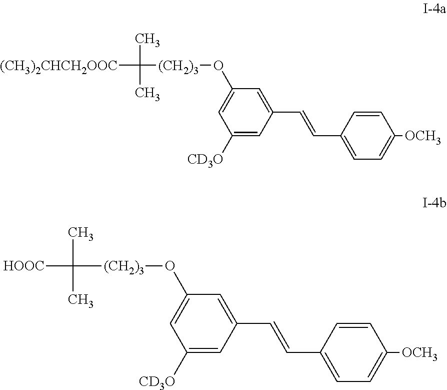

### Example 3.1

### 4′-methoxy-3,5-dihydroxy-(E)-stilbene (i-5)

2.0 g resveratrol was dissolved in 40 mL acetone, and then 3 g K₂CO₃ and 0.3 mL CH₃I were added successively. The reaction mixture was heated to reflux for 10 hours under nitrogen protection. After the heating was stopped, the reaction mixture was cooled down and filtered. The filter cake was washed with acetone, and the filtrate and the acetone washing solution were combined. The combined solution was concentrated, separated by 200-300 mesh silica gel column chromatography and eluted with a mixed solvent of petroleum ether:ethyl acetate:acetic acid (8.5:1.5:0.16) to obtain 1.1 g 4′-methoxy-3,5-dihydroxy-(E)-stilbene (i-5).

### Example 3.2

### 3-trideuterated methoxy-4′-methoxy-5-hydroxy-(E)-stilbene (i-6)

1.0 g i-5 was dissolved in 20 mL acetone, and 1 g K₂CO₃ and 0.15 mL CD₃I were added successively. The reaction mixture was heated to reflux for 10 hours under nitrogen protection. After the heating was stopped, the reaction mixture was cooled down and filtered. The filter cake was washed with acetone, and the filtrate and the acetone washing solution were combined. The combined solution was concentrated, separated by 200-300 mesh silica gel column chromatography and eluted with a mixed solvent of petroleum ether:ethyl acetate:acetic acid (8.5:1.5:0.16) to obtain 0.46 g 3-trideuterated methoxy-4′-methoxy-5-hydroxy-(E)-stilbene (i-6).

### Example 3.3

### 3-trideuterated methoxy-4′-methoxy-5-(4-methyl-4-isobutoxycarbonylpentyloxy)-(E)-stilbene (I-4a)

Referring to the method of reference example 1.2, i-6 was reacted with isobutyl 5-chloro-2,2-dimethylvalerate and the obtained reaction mixture was separated by silica gel column chromatography to obtain I-4a as a colorless oil with a yield of 52%.

### Example 3.4

### 3 -trideuterated methoxy-4′-methoxy-5-(4-methyl-4-carboxypentyloxy)-(E)-stilbene (I-4b)

Referring to the method of reference example 1.3, I-4a was reacted with LiOH to prepare I-4b with a yield of 75%. ¹H-NMR (d₆-DMSO, δ ppm): 1.12 (s, 6H), 1.62-1.69 (m, 4H), 3.76 (s, 3H); 3.96-3.99 (m, 2H), 6.38 (m, 1H), 6.95-7.03 (m, 3H), 7.22 (d, 1H), 7.55 (d, 2H), 12.15 (s, 1H).

### EXAMPLE 4

### Evaluation of Anti-Liver Fibrosis In Vivo

CCl₄ was prepared into a 40% CCl₄ oil with commercially available non-genetically modified peanut oil. The test compound was prepared into a 10 mg/ml solution with a 0.5% sodium carboxymethyl cellulose solution, for later use.

Adult male SD rats were randomly divided into a normal control group, a model group and a drug treatment group. The rats in the normal control group were intraperitoneally injected with peanut oil (0.3 ml/100 g, 1 time/3 days), and intragastrically administrated with 0.5% sodium carboxymethyl cellulose solution (0.5 ml/100 g, 1 time/1 day). The rats in the model group were intraperitoneally injected with 40% CCl₄ oil (0.3 ml/100 g, 1 time/3 days), and intragastrically administrated with 0.5% sodium carboxymethyl cellulose solution (0.5 ml/100 g, 1 time/day). The rats in the drug treatment group were intraperitoneally injected with 40% CCl₄ oil (0.3ml/100 g, 1 time/3 days), and intragastrically administrated with the 0.5% sodium carboxymethyl cellulose solution of the test compound (0.5 ml/100 g, 1 time/day). Rats in each group were bred in an air-conditioned animal laboratory with a temperature of (23±1)° C. and a relative humidity of (45±5)%, with day and night alternating every 12 hours, and they were free to eat and drink.

After being fed for 8 weeks, all the rats were fasted for 12 hours. After anesthesia, the blood of the rat was collected from the femoral artery, and the whole liver tissue was quickly weighed and frozen in liquid nitrogen for later use. 3 rats in each group were taken and their livers were cut, soaked into formalin solution for 24 h and prepared to paraffin-embedded pathological sections. The whole blood of the rats was centrifuged at 3000 r/min for 10min to obtain the separated serum, which was then frozen and stored for later use.

Alanine aminotransferase (ALT), aspartate aminotransferase (AST), alkaline phosphatase (ALP), and lactate dehydrogenase (LDH) in the serum were measured by Hitachi 7600 automatic biochemical analyzer. The results are shown in Table 1:

TABLE 1
Protection against CC1₄ induced liver injury in rats

| Groups | ALT (U/L) | AST (U/L) | ALP (U/L) | LDH (U/L) |
|---|---|---|---|---|
| Normal control group | 28.2 ± 7.2 | 103.7 ± 30.7 | 138.2 ± 29.4 | 615.4 ± 271.7 |
| Model group | 193.5 ± 124.3 | 458.6 ± 226.9 | 211.7 ± 108.0 | 882.0 ± 260.5 |
| Resveratrol | 96.8 ± 41.9 | 205.33 ± 85.3 | 203.6 ± 43.9 | 846.5 ± 261.2 |
| I-1b | 85.3 ± 46.4 | 209.1 ± 63.2 | 149.9 ± 52.9 | 843.1 ± 247.9 |
| I-2b | 69.1 ± 23.3 | 192.4 ± 78.4 | 141.6 ± 27.6 | 836.5 ± 375.5 |
| I-3b | 79.9 ± 43.8 | 221.9 ± 109.4 | 154.3 ± 65.0 | 780.4 ± 152.3 |
| I-4b | 91.3 ± 32.5 | 233.5 ± 90.6 | 140.8 ± 60.2 | 771.1 ± 210.2 |

The concentration of hydroxyproline (HYP) or malondialdehyde (MDA), and the activity of superoxide dismutase (SOD) or glutathione peroxidase(GSH-Px) in liver tissues were detected according to the corresponding operation steps of ELISA kit (Nanjing Jiancheng Biological Company). The results are shown in Table 2:

TABLE 2
Protection against CCl₄ induced liver fibrosis in rats

| | HYP | SOD | MDA | GSH-PX |
|---|---|---|---|---|
| Groups | (μg/mg protein) | (U/mg protein) | (nmol/mg protein) | (U/mg protein) |
| Normal control group | 0.42 ± 0.08 | 29.71 ± 3.77 | 0.75 ± 0.12 | 293.52 ± 33.11 |
| Model group | 0.96 ± 0.18 | 27.28 ± 4.57 | 1.26 ± 0.38 | 187.79 ± 41.82 |
| Resveratrol | 0.93 ± 0.15 | 28.09 ± 4.55 | 1.17 ± 0.35 | 174.33 ± 40.75 |
| I-1b | 0.59 ± 0.06 | 29.99 ± 5.24 | 0.72 ± 0.12 | 204.78 ± 27.75 |
| I-2b | 0.58 ± 0.29 | 33.60 ± 7.64 | 0.70 ± 0.08 | 231.48 ± 58.72 |
| I-3b | 0.70 ± 0.61 | 31.33 ± 6.50 | 0.92 ± 0.43 | 194.09 ± 32.82 |
| I-4b | 0.68 ± 0.27 | 28.64 ± 6.33 | 0.77 ± 0.14 | 224.33 ± 2.16 |

### EXAMPLE 5

### Evaluation of Anti-Pulmonary Fibrosis In Vivo

Bleomycin (batch number: 16037911, Hisun Pfizer Co., Ltd.) was prepared into a 5 mg/mL solution with normal saline; and the test compound was prepared into a solution with a 0.5% sodium carboxymethyl cellulose solution to a certain concentration for later use.

Adult male SD rats were randomly divided into a normal control group, a model group and a drug treatment group. After the animals in each group were anesthetized by intraperitoneal injection of 20% urethane (10 mL/kg), they were incised in the middle of the neck to expose the trachea. The rats in the normal control group were injected with 0.2 ml normal saline into the trachea all at once, and intragastrically administrated with 0.5% sodium carboxymethyl cellulose solution (0.5 ml/100 g, 1 time/day) the next day. The rats in the model group were injected with 0.2 ml of bleomycin solution into the trachea all at once, and simultaneously intragastrically administrated with 0.5% sodium carboxymethyl cellulose solution (0.5 ml/100 g, 1 time/day). The rats in the drug treatment group were injected with 0.2 ml bleomycin solution into the trachea all at once and simultaneously intragastrically administrated with the 0.5% sodium carboxymethyl cellulose solution of the test compound at different concentrations (0.5 ml/100 g, 1 time/day). The rats in each group were bred in an air-conditioned animal laboratory with a temperature of (23±1)° C. and a relative humidity of (45±5)%, with day and night alternating every 12 hours, and they were free to eat and drink. The rats were fasted for 12 hours on the 14th day after administration. After anesthesia, the blood of the rat was collected from the femoral artery and the lung tissue was separated. The right lung was perfused with 10% neutral formaldehyde through the trachea and then was put into stationary liquid for fixation and pathological examination. The left lung was cryopreserved in a refrigerator at -80° C. for testing the biochemical indicators.

The contents of HYP, MDA, GSH and SOD in lung tissues were detected according to the corresponding operation steps of ELISA kit (Nanjing Jiancheng Biological Company). The results are shown in Table 3:

TABLE 3
Protection against bleomycin-induced pulmonary fibrosis in rats

| | dose | HYP | MDA |
|---|---|---|---|
| Group | (mg/kg) | (μg/mg protein) | (nmol/mg protein) |
| Normal control group | 0 | 0.39 ± 0.07 | 0.65 ± 0.13 |
| Model group | 0 | 0.81 ± 0.13 | 1.08 ± 0.15 |
| Resveratrol | 100 | 0.75 ± 0.34 | 0.92 ± 0.33 |
| I-1b | 50 | 0.71 ± 0.17 | 0.91 ± 0.05 |
| | 100 | 0.54 ± 0.12 | 0.84 ± 0.09 |
| I-2b | 50 | 0.52 ± 0.11 | 0.75 ± 0.13 |
| | 100 | 0.45 ± 0.08 | 0.76 ± 0.06 |
| I-3b | 50 | 0.51 ± 0.09 | 0.81 ± 0.09 |
| | 100 | 0.47 ± 0.10 | 0.73 ± 0.06 |
| I-4b | 50 | 0.74 ± 0.19 | 0.96 ± 0.19 |
| | 100 | 0.59 ± 0.12 | 0.78 ± 0.24 |

### EXAMPLE 6

### Evaluation of Subacute Toxicity

Male ICR mice aged 6-8 weeks were fasted for 16 hours and randomly divided into groups, 5 mice per group. The test compound was formulated into a suspension with a 0.5% sodium carboxymethyl cellulose solution, and administered intragastrically at a concentration of 200 mg/kg, once a day for 4 consecutive weeks. After the last administration, the mice were fasted for 12 hours but were free to drink water during the fasting period. Then the mice were anesthetized. Blood was taken from the abdominal aorta and serum was prepared for the determination of blood biochemical and immunological indicators. EDTA anticoagulant blood was prepared for routine blood measurement. Except for some significant increase in transaminase (ALT, AST, ASP) of some drug administration groups, there were no significant differences in other indicators among the groups. The evaluation results are shown in Table 4:

TABLE 4
Effects of continuous administration of target compounds on liver function in mice

| Group | ALT (U/L) | AST | ALP |
|---|---|---|---|
| Normal control group | 18.18 ± 3.95 | 90.5 ± 19.44 | 215.33 ± 27.33 |
| I-1b | 47.33 ± 10.61 | 161.6 ± 18.32 | 471.35 ± 29.88 |
| I-2b | 26.47 ± 6.79 | 105.33 ± 16.85 | 242.83 ± 83.29 |
| I-3b | 36.57 ± 10.40 | 138.32 ± 29.72 | 381.67 ± 221.42 |
| I-4b | 16.07 ± 2.45 | 72.73 ± 15.82 | 258.33 ± 219.45 |

1. A resveratrol derivative of formula I or a pharmaceutically acceptable salt thereof: 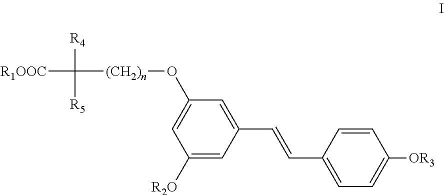
in formula I, R₁ is hydrogen or C₁-C₄ alkyl, R₂ and R₃ are each independently C₁-C₃ alkyl or C₁-C₃ deuterated alkyl, R₂ and R₃ are the same or different; the C₁-C₄ alkyl or C₁-C₃ alkyl is optionally substituted with one or more substituents each independently selected from the group consisting of halogen (for example, fluorine, chlorine, bromine, iodine), methyl, hydroxyl, ethyl, amino, methoxy, and nitro; R₄ and R₅ are each independently substituted or unsubstituted C₁-C₃ alkyl; and n is an integer of 0-5.

2. The resveratrol derivative according to claim 1, wherein the resveratrol derivative of formula I has the following formula I-0: 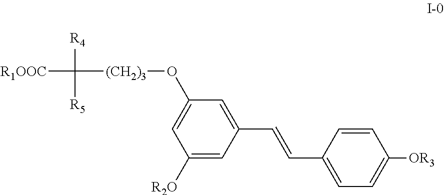
in formula I-0, R₁ is hydrogen or C₁-C₄ alkyl, R₂ and R₃ are each independently C₁-C₃ alkyl or C₁-C₃ deuterated alkyl, R₂ and R₃ are the same or different; the C₁-C₄ alkyl or C₁-C₃ alkyl is optionally substituted with one or more substituents each independently selected from the group consisting of halogen (for example, fluorine, chlorine, bromine, iodine), methyl, hydroxyl, ethyl, amino, methoxy, and nitro; and R₄ and R₅ are each independently substituted or unsubstituted C₁-C₃ alkyl.

3. The resveratrol derivative according to claim 1, wherein the resveratrol derivative of formula I has the following formula I-1: 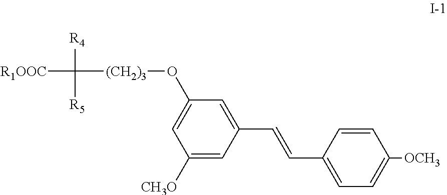
in formula I-1, R₁ is hydrogen or C₁-C₄ alkyl; wherein, the C₁-C₄ alkyl is optionally substituted with one or more substituents each independently selected from the group consisting of halogen (for example, fluorine, chlorine, bromine, iodine), methyl, hydroxyl, ethyl, amino, methoxy, and nitro; R₄ and R₅ are each independently substituted or unsubstituted C₁-C₃ alkyl.

4. The resveratrol derivative according to claim 1, wherein the resveratrol derivative of formula I has the following formula I-2: 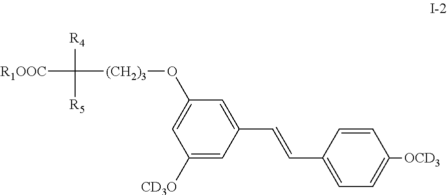
in formula I-2, R₁ is hydrogen or C₁-C₄ alkyl; wherein, the C₁-C₄ alkyl is optionally substituted with one or more substituents each independently selected from the group consisting of halogen (for example, fluorine, chlorine, bromine, iodine), methyl, hydroxyl, ethyl, amino, methoxy, and nitro; R₄ and R₅ are each independently substituted or unsubstituted C₁-C₃ alkyl.

5. The resveratrol derivative according to claim 1, wherein the resveratrol derivative of formula I has the following formula I-3: 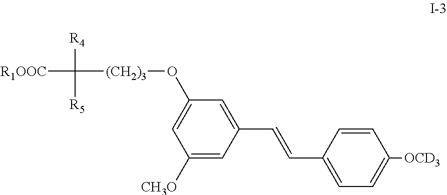
in formula I-3, R₁ is hydrogen or C₁-C₄ alkyl; wherein, the C₁-C₄ alkyl is optionally substituted with one or more substituents each independently selected from the group consisting of: halogen (for example, fluorine, chlorine, bromine, iodine), methyl, hydroxyl, ethyl, amino, methoxy, and nitro; R₄ and R₅ are each independently substituted or unsubstituted C₁-C₃ alkyl.

6. The resveratrol derivative according to claim 1, wherein the resveratrol derivative of formula I has the following formula I-4: 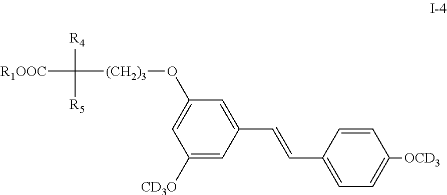
in formula I-4, R₁ is hydrogen or C₁-C₄ alkyl; wherein, the C₁-C₄ alkyl is optionally substituted with one or more substituents each independently selected from the group consisting of halogen (for example, fluorine, chlorine, bromine, iodine), methyl, hydroxyl, ethyl, amino, methoxy, and nitro; R₄ and R₅ are each independently substituted or unsubstituted C₁-C₃ alkyl.

7. The resveratrol derivative according to claim 1, wherein the resveratrol derivative of formula I has the structure as shown in the following table:

| | |
|---|---|
| 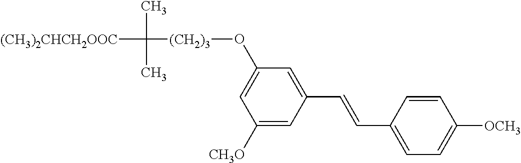 | I-1a |
| 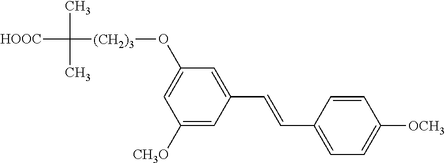 | I-1b |
| 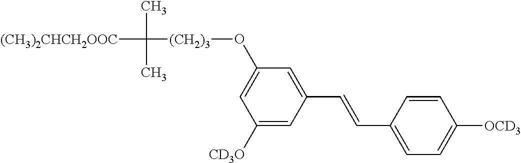 | I-2a |
| 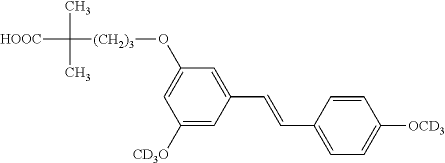 | I-2b |
| 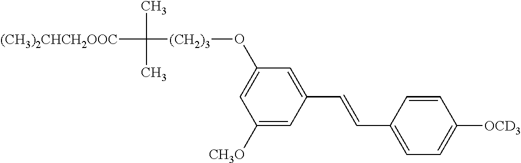 | I-3a |
| 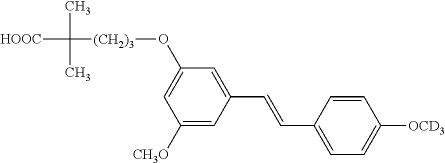 | I-3b |
| 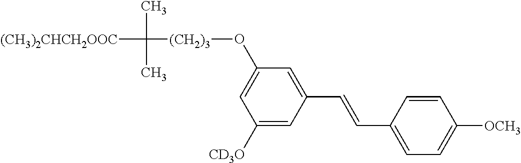 | I-4a |
| 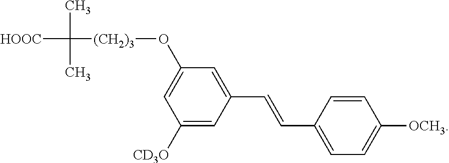 | I-4b |

8. A pharmaceutical composition, comprising a resveratrol derivative or a pharmaceutically acceptable salt thereof according to claim 1, and suitable pharmaceutical carriers or excipients.

9. The pharmaceutical composition according to claim 8, wherein the composition may be a solution, tablet, capsule or injection.

10. A method for treating a fibrotic disease in a subject in need thereof, comprising the step of administrating to the subject an effective amount of the resveratrol derivative or flail the pharmaceutically acceptable salt thereof according to claim 1.

11. The method according to claim 10, wherein the fibrotic disease is selected from the group consisting of liver fibrosis, lung fibrosis, kidney fibrosis and cardiac fibrosis.
